(19) United States
(12) Patent Application Publication
(10) Pub. No.: US 2004/0006336 A1
(43) Pub. Date: Jan. 8, 2004

(54) Apparatus and method for RF ablation into conductive fluid-infused tissue

(75) Inventor: David K. Swanson, Campbell, CA (US)

Correspondence Address: LYON & LYON LLP, 633 WEST FIFTH STREET, SUITE 4700, LOS ANGELES, CA, 90071, US

(73) Assignee: SCIMED Life Systems, Inc.

(21) Appl. No.: 10/188,487

(22) Filed: Jul. 2, 2002

(51) Int. Cl. A61B 18/14

(52) U.S. Cl. 606/41; 607/99; 607/105; 607/113

(57) ABSTRACT

A radiofrequency (RF) ablation device includes a cannula having a proximal end, a distal end, and a lumen extending therethrough. At least one electrode having a lumen and plurality of ports is disposed within the cannula. The electrode can reciprocate between a proximally retracted position and a distally extended position. The at least one electrode is coupled to a source of pressurized conductive fluid. The RF ablation device is used to pre-treat a region of tissue with a high-pressure injection of conductive fluid prior to the delivery of RF energy to the tissue. The pre-treatment step aids in creating extremely large lesions within the tissue.

### FIELD OF THE INVENTION

The field of the invention relates generally to devices and methods for the use of radio frequency electrosurgical probes for the treatment of tissue. More specifically, the present invention relates to an electrosurgical device having at least one hollow, tissue-penetrating electrode that is used to deliver a pressurized jet of conductive fluid to a region of tissue as well as provide RF energy to the fluid-infused tissue.

### BACKGROUND OF THE INVENTION

The delivery of radio frequency energy to target regions within solid tissue is known for a variety of purposes. Of particular interest to the present invention, radio frequency energy may be delivered to diseased regions in target tissue for the purpose of tissue necrosis. For example, the liver is a common depository for metastases of many primary cancers, such as cancers of the stomach, bowel, pancreas, kidney and lung. Electrosurgical probes for deploying multiple electrodes have been designed for the treatment and necrosis of tumors in the liver and other solid tissues.

Electrosurgical probes typically comprise a number of wire electrodes that are extended into a tissue region of interest from the distal end of a cannula. RF power is delivered to the wire electrodes to heat and necrose tissue within the region of target tissue. It is desirable to heat and necrose tissue within a precisely defined volumetric region of target tissue. One solution, for example, disclosed in U.S. Pat. No. 6,050,992, incorporated by reference as if set forth fully herein, uses a plurality of evenly spaced electrodes to that form a precisely defined array with the target tissue.

It is also desirable to have an electrosurgical probe that can create large, precisely defined lesions. While devices such as that disclosed in U.S. Pat. No. 6,050,992 may provide for precisely defined lesions, the ultimate size of the lesion may be limited by a number of factors. Generally, when RF energy is applied to an electrode, most of the RF energy (and heat) is delivered within a few millimeters of the ablation electrode. Lesion depth is extended by the thermal conduction of heat to deeper tissue layers over time (although some heating of the deeper tissue layers is produced by the RF energy). In order to prevent an explosive release of steam that can disrupt tissue and cause tissue perforations, it is preferable that local tissue temperatures not exceed 100° C. This requirement limits, to a certain extent, the power that is applied to each electrode. In addition, when tissue undergoes ablation, the impedance increases between the tissue and the electrode; thereby limiting the amount of power than can be applied to the tissue region of interest.

One technique that has been used to create deeper lesions is the irrigation and pumping of a saline solution directly into the tissue to be ablated. The irrigation is typically accomplished using hollow electrodes/needles that have holes drilled therein that allow saline solution to exit (at low pressure and flow rates) into the tissue of interest. These same needle-type structures are also used to deliver the RF energy during ablation. The injection of conductive fluid decreases electrical resistance (i.e., reduces ohmic losses) and thus permits the tissue to carry more energy without exceeding the 100° C. upper temperature limit. The difficulty with this method lies in the unpredictability of the fluid transfer. Moreover, prior art devices typically delivery saline solutions at relatively low pressures, relying on the migration of the saline fluid through the extracellular space. Consequently, it is sometimes difficult to produce deep penetration of saline solution over a specific portion of the tissue of interest.

For example, experimental results using injection by needle of dyed saline solution indicate that injectate tends to flow in between tissue layers and could orient current in unexpected directions from the injection site. The conductive fluid, in other words, does not reliably go in a consistent pattern thus making a predictable and precise ablation of tissue ablation very difficult.

It is desirable, therefore, to improve RF ablation techniques so that deeper lesions can be created of a predictable size while at the same time keeping tissue temperatures below 100° C. throughout the lesion area. As will be described in more detail below, the present invention provides improved lesion creation such that it achieves these and other desired results, which will be apparent from the description below to those skilled in the art.

### SUMMARY OF THE INVENTION

In a first aspect of the invention a radiofrequency ablation device includes a cannula having a proximal end, a distal end, and a lumen extending therethrough. A plurality of pre-shaped electrodes are disposed in the cannula lumen to reciprocate between a proximally retracted position and a distally extended position. The plurality of electrodes include a lumen extending through at least a portion therethrough and a plurality of ports provided along at least a portion of each of the plurality of electrodes. A source of pressurized conductive fluid is coupled to the lumens of the plurality of electrodes. In the proximally retracted position all of the plurality of electrodes are radially constrained within the lumen of the cannula. In the distally extended position all of the plurality of electrodes deploy radially outward.

In a second separate aspect of the invention, a radiofrequency ablation device includes a cannula having a proximal end, a distal end, and a lumen extending therethrough. An electrode is disposed in the cannula lumen to reciprocate between a proximally retracted position and a distally extended position. The electrode includes a lumen extending through at least a portion therethrough. The electrode also includes a plurality of ports provided along at least a portion of the length of the electrode. A source of pressurized conductive fluid is coupled to the electrode lumen.

In a third aspect of the invention a method of performing radiofrequency ablation on tissue comprising the steps of positioning a radiofrequency ablation device within a region of tissue, deploying at least one electrode within the region of tissue, injecting, under pressure, a conductive fluid into the region of tissue with the at least one electrode, and delivering RF power to the region of tissue using the at least one electrode.

It is an object of the invention to provide an RF ablation device that can pre-treat tissue using high-pressure injection of a conductive fluid. This same device can also deliver RF energy to the injected tissue. It is a further object of the invention to provide a device and method that can make extremely large lesions in tissue using a combination of conductive fluid injection and RF ablation. Additional objects and advantages of the invention are described below.

### BRIEF DESCRIPTION OF THE DRAWINGS

FIG. 1(a) is a sectional view of a radiofrequency ablation device according to one preferred embodiment of the invention.

FIG. 1(b) is a cross-sectional view taken along the line A-A′ of the RF ablation device shown in FIG. 1(a).

FIG. 2(a) is a sectional view of a radiofrequency ablation device according to another preferred embodiment of the invention.

FIG. 2(b) is a cross-sectional view taken along the line B-B′ of the RF ablation device shown in FIG. 2(a).

FIG. 3(a) shows an electrode with a plurality of ports according to one embodiment of the invention.

FIG. 3(b) is a cross-sectional view taken along the line C-C′ of the RF ablation device shown in FIG. 3(a).

FIG. 4 shows a radiofrequency ablation device according to one preferred embodiment of the invention entering a treatment region TR of tissue T.

FIG. 5(a) is a partial sectional view of the distal end of the cannula of an RF ablation device according to another embodiment of the invention.

FIG. 5(b) is a cross-sectional view taken along the line D-D′ of the RF ablation device shown in FIG. 5(a).

FIG. 6 shows a radiofrequency ablation device according to another preferred embodiment of the invention entering a treatment region TR of tissue T.

FIG. 7(a) is a schematic view of a RF ablation device shown connected to a pump and reservoir.

FIG. 7(b) is a schematic view of an alternative RF ablation device wherein the electrode is in a loop-type configuration and a pump is attached at both ends.

FIG. 8 shows an enlarged view of the distal region of an RF ablation device having a centrally disposed temperature probe.

FIG. 9 shows a radiofrequency ablation device according to yet another preferred embodiment of the invention entering a treatment region TR of tissue T.

### DETAILED DESCRIPTION OF THE PREFERRED EMBODIMENTS

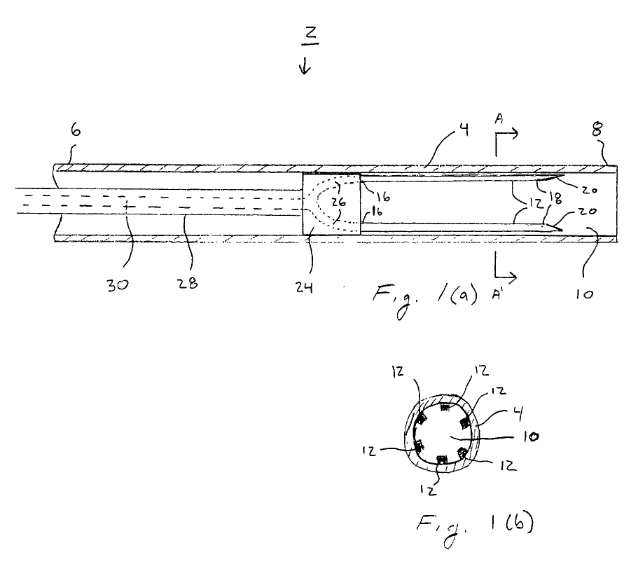

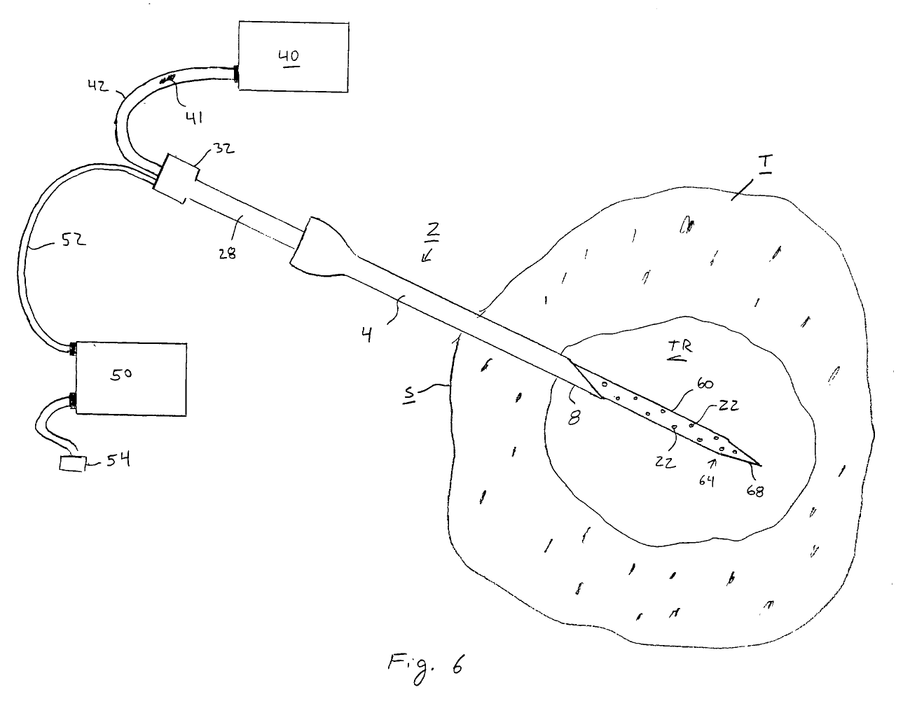

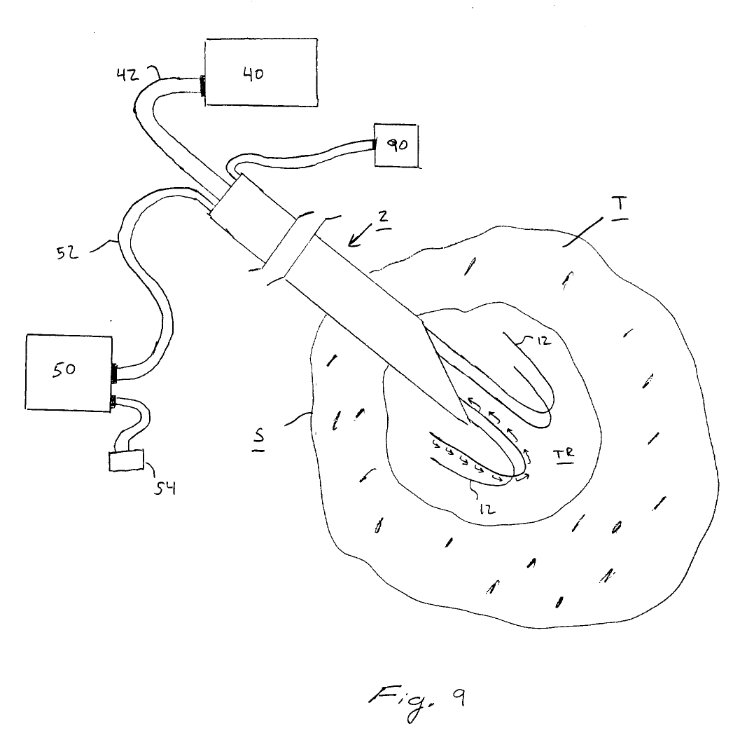

FIGS. 1(a) and 1(b) illustrate a radiofrequency (RF) ablation device 2 according to one preferred embodiment of the invention. The RF ablation device 2, which may take the form of a probe, includes a cannula 4 having a proximal end 6, a distal end 8, and a lumen 10 extending therethrough. The cannula 4 is preferably rigid or semi-rigid and is formed from metal, plastic, or some other rigid material. In some cases, the cannula 4 will have a sharpened tip at the distal end 8 to facilitate introduction to the tissue target site. FIGS. 6 and 9 show cannulas 4 having sharpened tips at their distal ends 8. In a preferred aspect of the invention, the cannula 4 is in the form of a hollow needle.

FIGS. 1(a) and 1(b) also show a plurality of electrodes 12 that are contained within the lumen 10 of the cannula 4. The electrodes 12 are preferably formed from a resilient material and are pre-shaped to form a specific shape once the electrodes 12 are released from the confines of the cannula 4. In one preferred aspect, the electrodes 12 are formed from stainless steel hypotube. The cannula 4 serves to constrain the individual electrodes 12 in a radially collapsed configuration to facilitate their introduction to the tissue target site. The electrodes 12 can then be deployed to their desired configuration, usually a three-dimensional configuration, by extending the distal ends of the electrodes 12 from the distal end 8 of the cannula 4 into the tissue. In this manner, the electrodes 12 are reciprocable within the cannula 4. Deployment of the electrodes 12 may be accomplished by pushing the electrodes 12 out of the distal end 8 of the cannula 4 or, alternatively, retraction of the cannula 4 while leaving the electrodes 12 in place. During deployment of the electrodes 12, when the electrodes 12 emerge beyond the distal end 8 of the cannula 4 they begin to deflect (as a result of their own spring or shape memory) in a radially outward pattern.

FIG. 1(b) shows six electrodes 12 being used in the RF ablation device 2, however, a larger or smaller number of electrodes 12 can also be used in accordance with the invention. For example, as few as three or as many as twelve can be used with the RF ablation device 2. FIG. 1(b) also shows that the electrodes 12 are equally spaced from one another. This construction is preferred because it creates a symmetrical array of electrodes 12 upon deployment. The symmetrical array produces a symmetrical lesion.

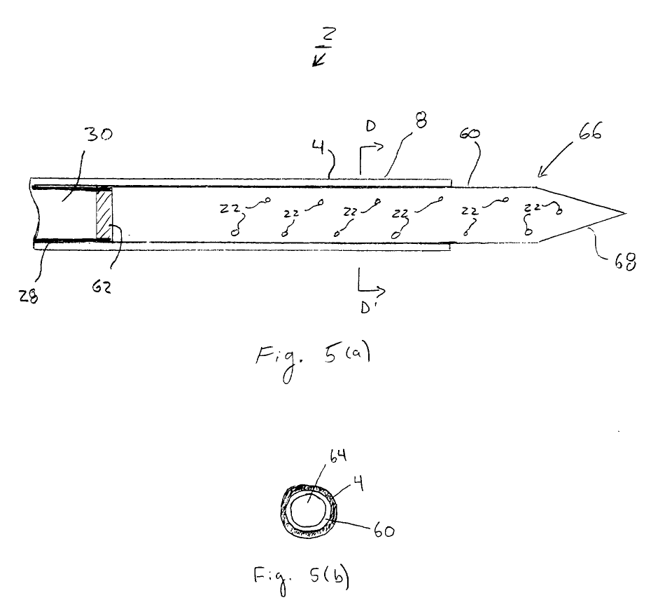

Referring to FIG. 1(a), the electrodes 12 are attached at their proximal ends to a hub 24. The hub 24 includes a series of flowpaths 26 that communicate with the lumen 14 of each electrode 12. The hub 24, in turn, is connected to a shaft 28 that includes a lumen 30 therethrough. The lumen 30 of the shaft 28 communicates with the lumen 14 of each electrode 12 via the flowpaths 26 in the hub 24. The shaft 28 can include a handle portion 32 (as is shown in FIGS. 5 and 6) that an operator holds during the delivery of the electrodes 12 to the tissue region of interest.

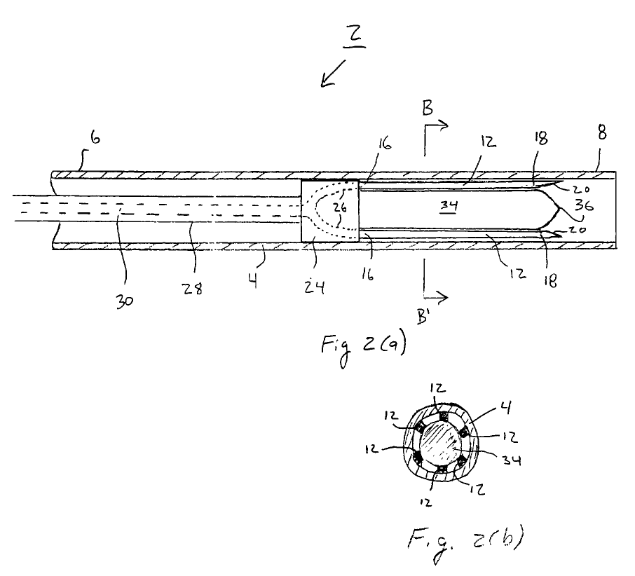

FIGS. 2(a) and 2(b) show an alternative embodiment of the invention. In this embodiment, a core member 34 is disposed coaxially within the cannula 4 and radially inward of the electrodes 12. In this embodiment, the electrodes 12 are constrained between the circumferential surface of the core member 34 and the inner surface of the cannula lumen 10. The core member 34 may contain one or more channels (not shown) that receive individual electrodes 12 to assist in the accurate deployment of the electrodes 12. Preferably, the core member 34 moves with the electrodes 12 when the shaft 28 is advanced/retracted. The core member 34 can also enter the tissue at the same time as the electrodes 12. The core member 34 may include a sharpened distal tip 36 that aids in penetrating tissue. The core member 34 may be electrically coupled to the electrodes 12 (in which case it acts as an additional electrode of the same polarity as the electrodes 12) or may be electrically isolated from the electrodes 12. When the core member 34 is electrically isolated, it can remain neutral during RF delivery, on alternatively, it may be energized in the opposite polarity and this act as a return electrode in a bipolar treatment protocol.

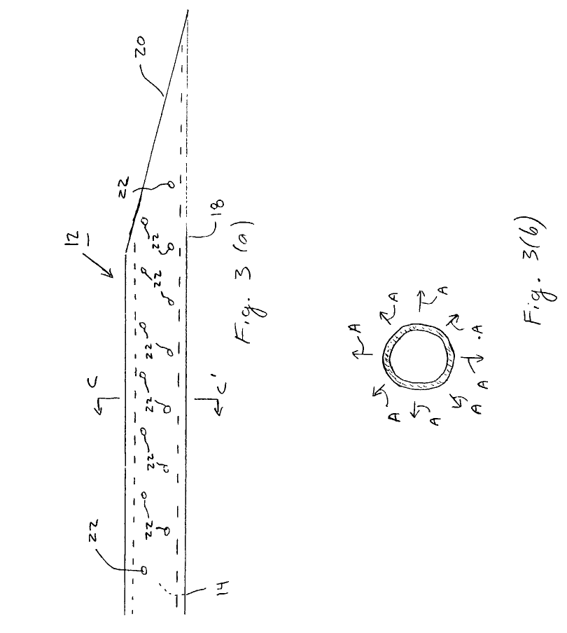

Referring now to FIGS. 3(a) and 3(b), the electrodes 12 have a lumen 14 that extends a portion of the way through each electrode 12. Preferably, the lumen 14 extends from a proximal end 16 of the electrode 12 to a distal region 18 of the electrode 12. The distal-most tip of the electrode 12 is sealed. Preferably, as shown in FIGS. 1(a), 2(a), and 3(a), the distal region 18 of the electrode 12 terminates in a sharpened tine 20. The sharpened tines 20 help the electrodes 12 penetrate the tissue.

The electrodes 12 include a plurality of ports 22 that are drilled into the circumferential surface of the electrodes 12. The ports 22 provide access to the lumen 14 of the electrode 12. The ports 22 can be formed by laser drilling or other commonly known techniques used to form small holes in rigid materials. Preferably, there are between about 20 to about 40 ports 22 on each electrode 12. In a preferred aspect of the invention the ports 22 have a diameter within the range of about 0.002″ to about 0.004″. FIGS. 3(a) and 3(b) show a series of ports 22 around the entire circumference of the electrode 12. In this manner, conductive fluid (discussed in detail below) can be ejected in a full 360° around the electrode 12. Arrows A in FIG. 3(b) show the flow direction of the conductive fluid. It is also possible that some procedures may require the ports 22 to be located in only a specific region or regions of the electrode 12 (for example, only on one side of the electrode 12). This would allow the directed application of conductive fluid to the tissue region of interest.

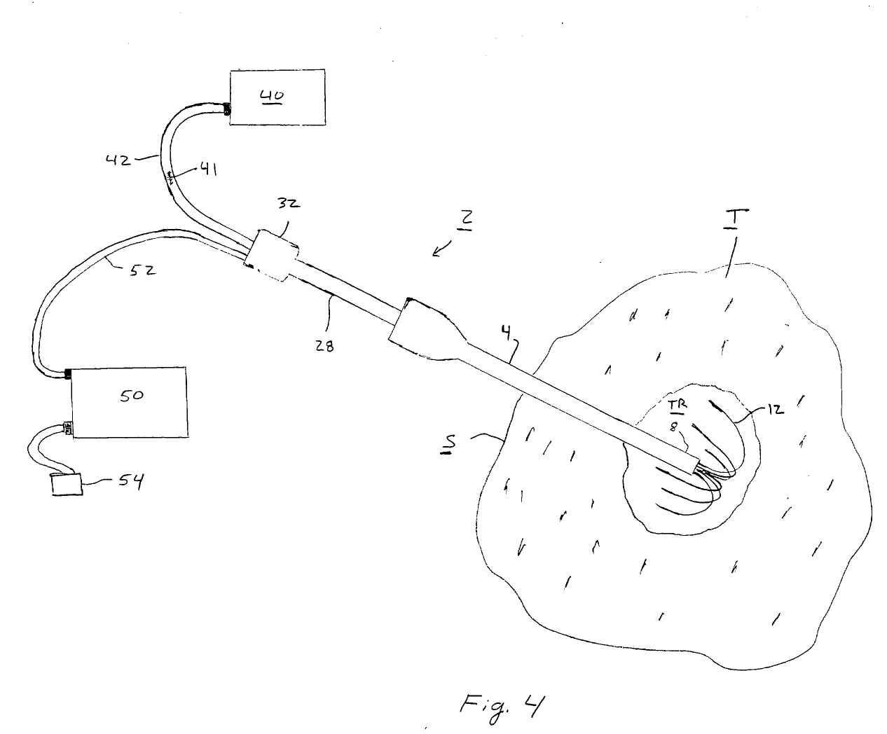

Referring now to FIG. 4, the RF ablation device 2 is coupled to a pressurized source of conductive fluid 40. The pressurized source of conductive fluid 40 delivers conductive fluid 41 (shown in FIGS. 4 and 6) to the lumen 30 of the shaft 28 via tubing 42. The conductive fluid 41 passes through the flowpaths 26 of the hub 24 and into the lumen 14 of each electrode 12. The pressurized source of conductive fluid 40 preferably produces a pressure within the range of about 1000 psi to about 2000 psi in the proximal end of the electrodes 12 and a pressure within the range of about 500 psi to about 1500 psi at the electrode ports 22. The pressurized conductive fluid 41 is ejected out the ports 22 and into the tissue target site as a series of small jets of conductive fluid 41.

The conductive fluid 41 can comprise any number of electrically conductive solutions including, but not limited to, saline (NaCl), potassium chloride (KCI), sodium bicarbonate (NaHCO₃), sodium citrate (Na₃C₆H₅O₇), potassium citrate (K₃C₆H₅O₇), ionic radiographic contrast materials such as, for example, RENOGRAFIN, and the like. The concentration of the conductive fluid 41 is chosen to produce an ohmic resistivity within the range of about 2 ohm-cm to about 100 ohm-cm. Preferably, a conductive fluid 41 with a low ohmic resistivity is used. Consequently, higher concentrations of the exemplary salt solutions are needed to produce the low ohmic resistivity. For example, a 20% NaCl salt solution (wt/volume) has a resistivity of about 2 ohm-cm.

Still referring to FIG. 4, the RF ablation device 2 is also coupled to a radiofrequency generator 50. The RF generator 50 delivers radiofrequency current via a cable 52 that connects to each electrode 12. The RF current may be applied in a monopolar or biopolar fashion. The RF generator 50 may optionally be used to deliver a first “deployment” current to facilitate passage of the electrodes 12 through the tissue. A second, “ablation” current can then be used to ablate the tissue.

In monopolar operation, as is shown in FIGS. 4 and 6, a passive or dispersive electrode 54 is provided to complete the return path for the circuit that is created. Such electrodes, which will usually be attached externally to the patient's skin, will have a much larger area, typically about 130 cm² for an adult so that current flux is sufficiently low to avoid significant heating and other biological effects. It may also be possible to provide the dispersive return electrode 54 directly on the cannula 4 or core member 34.

Still referring to FIG. 4, a treatment region TR within tissue T is located beneath the skin or an organ surface S of a patient. The treatment region TR may be a tumor where it is desired to treat the tissue by RF ablation. To access the treatment region TR, the RF ablation device 2 is advanced into the tissue T so that the distal end 8 of the cannula 4 is within the treatment region TR. The cannula 4 can be sharpened at its tip, for example, as is shown in FIG. 6, and directly inserted into the tissue. Alternatively, a separate sheath (not shown) may be introduced through the skin or organ surface S to provide access for the RF ablation device 2. After the cannula 4 is properly placed, the shaft 28 is advanced distally to deploy the electrodes 12 radially outward from the distal end 8 of the cannula 4. The shaft 28 is preferably advanced to cause the electrodes 12 to fully evert in order to substantially circumscribe the treatment region TR. Alternatively, the shaft 28 can remain in place while the cannula 4 is retracted in the proximal direction. The delivery of the RF ablation device 2, including the cannula 4 and electrodes 12 can preferably be monitored using conventional imaging techniques such as ultrasonic scanning, magnetic resonance imaging (MRI), computer-assisted tomography (CAT), flouroscopy, nuclear scanning, and the like.

Upon deployment of the electrodes 12, the pressurized source of conductive fluid 40 is allowed to communicate with the lumen 30 of the shaft 28 (through appropriate valve mechanisms or the like). The conductive fluid 41 passes into the lumen 14 of each electrode 12 and is ejected out of the ports 22 under high pressure. The conductive fluid 41 is pressure injected into the treatment region TR for a period of time, which may be within the range of about 100 milliseconds to about 2 seconds.

After the treatment region TR has been injected with conductive fluid 41, the RF generator 50 delivers radiofrequency current to the fluid-injected treatment region TR. Typically, the power and amount of time that the RF current is delivered to the patient is programmed by the operator into the RF generator 50. The combination of the high pressure injection of conductive fluid 41 with the subsequent delivery of RF current is able to create extremely large lesions in the treatment region TR that are much larger than the lesions formed with just standard RF ablation.

FIGS. 5(a) and 5(b) show an alternative embodiment of the RF ablation device 2. In this embodiment, a single electrode 60 is used to both deliver the conductive fluid 41 and the RF energy. Preferably, the single electrode 60 is in the form of a hollow, closed end needle having an internal diameter of about 2 mm although other sizes may be used in accordance with the invention. This single electrode 60 is reciprocable within the lumen of a cannula 4 and is shown in FIG. 5(a) connecting via a connecting member 62 to a shaft 28 having a lumen 30 therein for passage of conductive fluid 41. Alternatively, the shaft 28 and connecting member 62 can be removed entirely, and the electrode 60 itself would be connected to the pressurized source of conductive fluid 40. In this alternative construction, a portion of the proximal exterior portion of the electrode 60 would have to be insulated to protect the operator from receiving RF energy when holding the electrode 60.

The single electrode 60 contains a lumen 64 therethrough (shown in FIG. 5(b)) for the passage of conductive fluid 41. The lumen 64 passes through a portion of the electrode 60 and is sealed at its distal end 64. The electrode 60 preferably has a sharpened tip 68 that aids in penetrating tissue. The electrode 60 also contains a plurality of ports 22 on its circumferential exterior. The ports 22 preferably have diameters within the range of about 0.002″ to about 0.004″. The ports 22 are preferably evenly spaced around the circumference of the electrode 60 such that there is a linear separation of about 5 mm between adjacent ports 22. Preferably, there are about six lines (shown in FIG. 5(a)) of ports 22 about the circumference of the electrode 60 although more or less can be used and still fall within the scope of the invention.

FIG. 6 shows the above-described RF ablation device 2 being inserted into a tissue region of interest. As with the multiple electrode embodiment shown in FIG. 4, the RF ablation device 2 is coupled to a pressurized source of conductive fluid 40 via tubing 42. The conductive fluid 41 is delivered into the lumen 64 of the electrode 60 under high pressure. Preferably, the pressure within the proximal end of the electrode 60 is within the range of about 1000 psi to about 2000 psi while the pressure at the ports 22 is within the range of about 500 psi to about 1500 psi. The conductive fluid 41 is ejected out of the ports 22 and into the tissue target site in the form of a plurality of “jets” of conductive fluid 41. The conductive fluid 41 can comprise any number of conductive solutions including those identified above with respect to the multiple electrode embodiment.

Still referring to FIG. 6, The RF ablation device 2 is coupled to a radiofrequency generator 50. The RF generator 50 delivers radiofrequency current via a cable 52 to the single electrode 60. As with the multiple electrode embodiment, the RF generator 50 may optionally use a first “deployment” current to facilitate passage of the electrode 60 through the tissue. A second “ablation” current can then be applied to form the lesion. A passive or dispersive electrode 54 is provided to complete the return path for the circuit.

Operation of the RF ablation device 2 shown in FIG. 6 is similar to the operation of the RF ablation device 2 shown in FIG. 4 with the exception being there is no deployment of multiple electrodes. The treatment region TR is accessed by advancing the ablation device 2 into the tissue T so that the distal end 8 of the cannula 4 is within the treatment region TR. FIG. 6 shows a sharpened cannula 4 that is used to aid in delivering reaching the treatment region TR. As an alternative to direct insertion of the cannula 4, a separate sheath or the like may be introduced through the skin or organ surface S to provide access for the RF ablation device.

When the cannula 4 is properly positioned, the shaft 28 is pushed in the distal direction to advance the electrode 60 from the distal end 8 of the cannula 4. Alternatively, the shaft 28 can remain in place while the cannula 4 is retracted in the proximal direction. If the RF ablation device 2 does not use a separate shaft 28, then the electrode 60 is simply advanced into position within the cannula 4 or sheath. The delivery of the RF ablation device 2 can be monitored using conventional imaging techniques described in detail above.

After the electrode 60 has been moved into position, conductive fluid 41 is pumped into the lumen 64 of the electrode 60 from the source of pressurized conductive fluid 40. The conductive fluid 41 passes into the lumen 10 of the electrode 60 and is ejected out of the ports 22 under high pressure. The conductive fluid 41 is pressure-injected into the treatment region TR for a period of time, which may be within the range of about 100 milliseconds to about 2 seconds.

After the treatment region TR has been injected with conductive fluid, the RF generator 50 delivers radiofrequency current to the injected treatment region TR. The combination of the high-pressure injection of conductive fluid 41 with the subsequent delivery of RF current produces extremely large lesions with the tissue. One advantage of the RF ablation device 2 with the single electrode 60 is that the device has a much simpler construction than its multiple electrode counterpart. In addition, it is much easier to deploy the single electrode 60 to the region of interest than to deploy a plurality of smaller electrodes 60.

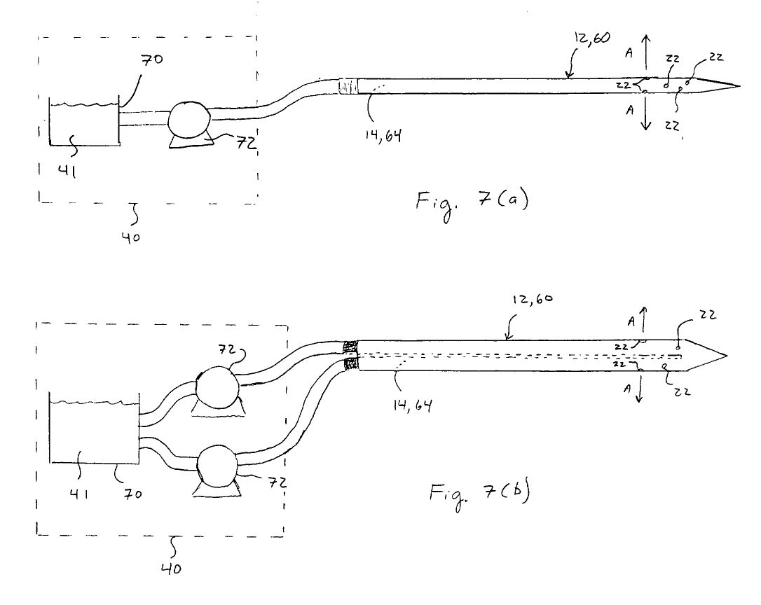

FIG. 7(a) shows one preferred manner of producing the pressurized source of conductive fluid 40. A reservoir 70 containing the conductive fluid 41 is connected to a pump 72. The pump 72 provides conductive fluid 41 to the electrode lumen 14, 64 at high pressure. The pump 72 preferably creates a high pressure within the lumen 14, 64 at the distal end 18, 66 of the electrodes 12, 60 such that narrow streams (shown by arrows A in FIGS. 7(a) and 7(b)) of conductive fluid 41 are ejected out of the ports 22. Because the electrode lumen 14, 64 is narrow, a substantial pressure drop is created along the length of the electrode 12, 60. To reduce this pressure drop, the internal lumen 14, 64 of the electrode 12, 60 may be formed into a loop-type of structure with both ends of the loop being pressurized. This alternative embodiment is illustrated in FIG. 7(b).

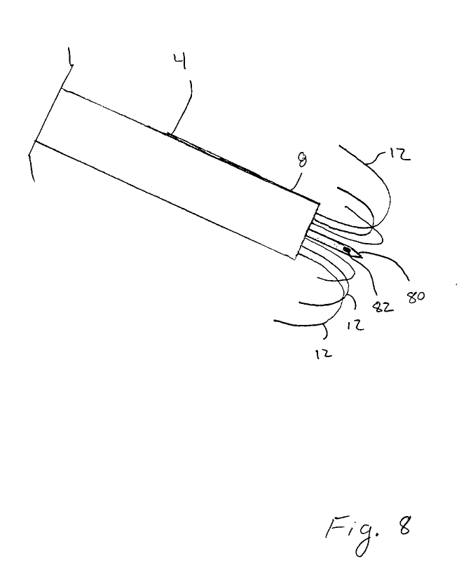

FIG. 8 shows an embodiment of the RF ablation device 2 using multiple electrodes 12. In this embodiment, a temperature probe 80 projects from the distal tip of a cannula 4 along with the plurality of electrodes 12. The temperature probe 80 includes a temperature sensor 82 that is used to detect the temperature of the tissue undergoing RF ablation. The temperature probe 80 may be formed on or in connection with the core member 34 if a core member 34 is used. The temperature sensor 82 can be any commonly known temperature sensor, such as a thermistor, thermocouple, or IC (digital) temperature sensor. Preferably, the temperature probe 80 and sensor 82 are located centrally to the deployed plurality of electrodes 12. The measured temperature is reported back to a monitoring device (not shown) which can then be displayed for the operator. The measured temperature readings can be used to determine the effectiveness of the ablation procedure when RF power is delivered to the electrodes 12. In another aspect, the temperature readings can be reported back to the RF generator 40 as means for controlling the amount of power delivered to the electrodes 12. If, for example, the temperature is rising at too fast a rate or exceeds a pre-determined set point, appropriate control circuitry (not shown) is triggered within the RF generator 40 to reduce the amount of RF current delivered to the electrodes 12.

FIG. 9 shows yet another embodiment of the invention. In this embodiment, the RF ablation device 2 uses conductive fluid 41 for two purposes. First, The RF ablation device 2 uses the conductive fluid 41 to pre-treat the tissue prior to RF ablation. The tissue is pre-treated by the high-pressure injection of conductive fluid 41 out of the ports 22 in the electrodes 12. This is the procedure discussed above with respect to the RF ablation devices shown in FIGS. 4 and 6.

In this embodiment, however, the conductive fluid 41 is also used to provide some amount of cooling during the relatively long RF ablation period. In this regard, the conductive fluid 41 is pumped through the electrodes 12 to irrigate the tissue T using the ports. Infusion rates as small as 1.0 ml/minute would significantly reduce the temperatures produced adjacent to the electrodes 12 at a fixed RF power, thereby enabling more power delivery to the tumor mass. This same procedure can be employed in the RF ablation device 2 using the single electrode 60.

An optional vacuum source 90, as seen in FIG. 9, may be coupled to the cannula 4 or sheath. The vacuum source 90 serves to collect the small volume of conductive fluid 41 used to irrigate the tissue T. During irrigation, the conductive fluid 41 preferentially travels along the electrode tracks (shown by the arrows in FIG. 9) back to the distal end 8 of the cannula 4 or sheath. The vacuum source 90 then withdraws this “pooled” conductive fluid 41 out of the tissue T.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

What is claimed is:

1. A radiofrequency ablation device comprising:
a cannula having a proximal end, a distal end, and a lumen extending therethrough;
a plurality of pre-shaped electrodes disposed in the cannula lumen to reciprocate between a proximally retracted position and a distally extended position, the plurality of electrodes including a lumen extending through at least a portion therethrough, the plurality of electrodes further including a plurality of ports provided along at least a portion of each of the plurality of electrodes;
a source of pressurized conductive fluid coupled to the electrode lumens; and
wherein in the proximally retracted position all of the plurality of electrodes are radially constrained within the lumen of the cannula and wherein in the distally extended position all of the plurality of electrodes deploy radially outward.

2. The radiofrequency ablation device of claim 1, wherein the plurality of electrodes includes at least three electrodes.

3. The radiofrequency ablation device of claim 1, further comprising a core disposed coaxially within the cannula and radially inward from the plurality of electrodes.

4. The radiofrequency ablation device of claim 3, wherein the core is reciprocable with the plurality of electrodes.

5. The radiofrequency ablation device of claim 1, wherein the source of pressurized fluid produces a pressure at the proximal end of the electrodes within the range of about 1000 psi to about 2000 psi.

6. The radiofrequency ablation device of claim 1, wherein the source of pressurized fluid produces a pressure at the ports of the electrodes within the range of about 500 psi to about 1500 psi.

7. The radiofrequency ablation device of claim 1, wherein the plurality of electrodes comprise stainless steel hypotube.

8. The radiofrequency ablation device of claim 1, wherein each electrode contains between 20 and 40 ports.

9. The radiofrequency ablation device of claim 1, wherein the plurality of ports have an internal diameter within the range of about 0.002″ to about 0.004.″

10. The radiofrequency ablation device of claim 1, further comprising a temperature probe having a temperature sensor located centrally to the plurality of electrodes.

11. The radiofrequency ablation device of claim 1, wherein the conductive fluid is saline.

12. The radiofrequency ablation device of claim 1, further comprising a radiofrequency generator connected to the plurality of electrodes.

13. The radiofrequency ablation device of claim 1, further comprising a source of vacuum coupled to the lumen of the cannula.

14. The radiofrequency ablation device of claim 1, wherein the plurality of ports are disposed around the entire circumference of at least one electrode.

15. A radiofrequency ablation device comprising:
a cannula having a proximal end, a distal end, and a lumen extending therethrough;
an electrode disposed in the cannula lumen to reciprocate between a proximally retracted position and a distally extended position, the electrode including a lumen extending through at least a portion therethrough, the electrode further including a plurality of ports provided along at least a portion of the length of the electrode;
a source of pressurized conductive fluid coupled to the electrode lumen.

16. The radiofrequency ablation device of claim 15, wherein the source of pressurized fluid produces a pressure at the proximal end of the electrode within the range of about 1000 psi to about 2000 psi.

17. The radiofrequency ablation device of claim 15, wherein the source of pressurized fluid produces a pressure at the ports of the electrode within the range of about 500 psi to about 1500 psi.

18. The radiofrequency ablation device of claim 15, wherein the electrode comprises a closed end, hollow needle.

19. The radiofrequency ablation device of claim 18, wherein the closed end, hollow needle has an internal diameter within the range of about 2 mm to about 3 mm.

20. The radiofrequency ablation device of claim 18, wherein adjacent ports are separated by a distance of about 5 mm.

21. The radiofrequency ablation device of claim 18, wherein the ports are spaced evenly around the circumference of at least a portion of the closed end, hollow needle.

22. The radiofrequency ablation device of claim 15, wherein the conductive fluid is saline.

23. The radiofrequency ablation device of claim 15, further comprising a radiofrequency generator connected to the plurality of electrodes.

24. The radiofrequency ablation device of claim 15, further comprising a source of vacuum coupled to the lumen of the cannula.

25. A method of performing radiofrequency ablation on tissue comprising the steps of:
positioning a radiofrequency ablation device within a region of tissue;
deploying at least one electrode within the region of tissue;
injecting, under pressure, a conductive fluid into the region of tissue with the at least one electrode; and
delivering RF power to the region of tissue using the at least one electrode.

26. The method of claim 25 further comprising the step of irrigating the region of tissue with a conductive fluid when RF power is applied.
